(19) United States
(12) Patent Application Publication
(10) Pub. No.: US 2016/0295995 A1
(43) Pub. Date: Oct. 13, 2016

(54) Lotion Applicator

(71) Applicant: Douglas Peterson, American Fork, UT (US)

(72) Inventor: Douglas Peterson, American Fork, UT (US)

(21) Appl. No.: 15/091,692

(22) Filed: Apr. 6, 2016

Related U.S. Application Data
(60) Provisional application No. 62/144,377, filed on Apr. 8, 2015.

(51) Int. Cl. A45D 40/26; A61F 13/40

(52) U.S. Cl. CPC A45D 40/26; A61M 35/006

(57) ABSTRACT

A lotion applicator for allowing a user to apply any of a variety of lotions, creams, medications, or ointments to various portions of the user's body without the assistance of another person. The lotion applicator includes an elongated strap of material having a first end and a second end. The first and second ends each have a handle thereon so that a user can hold an end of the elongated strap in each hand. A central portion of the elongated strap is configured to receive a lotion or cream thereon for application to a body part of the user, such as the user's back.

### CROSS REFERENCE TO RELATED APPLICATION

This application claims the benefit of U.S. Provisional Application No. 62/144,377 filed on Apr. 8, 2015. The above identified patent application is herein incorporated by reference in its entirety to provide continuity of disclosure.

### BACKGROUND OF THE INVENTION

### Field of the Invention

The present invention relates to lotion applicators. More specifically, the present invention provides a lotion applicator that allows a user to apply an ointment, cream, medication, or lotion to various portions or his or her body, such as the user's back, without the assistance of another person.

It is often necessary to apply a cream, lotion, ointment, oil, medication, or other similar substance, herein referred to simply as “lotion,” to a user's body in order to treat or protect the user's skin. For example, it is important to apply sun screen thoroughly before spending time outdoors in the sun. However, it can be difficult for a user to reach certain parts of his or her body, such as the user's back. As a result, the user may be unable to properly apply sun screen to his or her back. Further, it may be inconvenient to ask another person to help apply sun screen or another lotion, and there may not always be another person available to help apply the lotion.

Devices have been disclosed in the prior art that relate to sun screen application devices. These include devices that have been patented and published in patent application publications. These devices generally relate to wipes having lotion thereon for applying self-tanner or other lotions to the user's body, such as U.S. Pat. No. 5,972,360, U.S. Published Patent Application Number 2010/0034759, U.S. Published Patent Application Number 2006/0078515, U.S. Pat. No. 6,723,306, and U.S. Published Patent Application Number 2004/0228811.

These prior art devices have several known drawbacks. Devices in the prior art provide only specific lotion or tanning formulations for application to the user's skin and do not provide lotion applicators for helping the user to apply a lotion. Further, such devices do not assist the user in reaching hard to reach areas, such as the user's back in order to apply the lotion thereto. Thus, a lotion applicator that helps the user to apply lotions to his or her body is provided.

In light of the devices disclosed in the prior art, it is submitted that the present invention substantially diverges in design elements from the prior art and consequently it is clear that there is a need in the art for an improvement to existing lotion applicator devices. In this regard the instant invention substantially fulfills these needs.

### SUMMARY OF THE INVENTION

In view of the foregoing disadvantages inherent in the known types of lotion applicators now present in the prior art, the present invention provides a new lotion applicator wherein the same can be utilized for providing convenience for the user when applying any of various lotions to his or her body without the assistance of another person.

It is therefore an object of the present invention to provide a new and improved lotion applicator comprising an elongated strap having a handle at a first end and second end thereof, wherein the elongated strap is adapted to receive a lotion thereon.

It is another object of the present invention to provide a lotion applicator comprising an elongated strap having a substantially rectangular configuration.

Another object of the present invention is to provide a lotion applicator comprising an elongated strap impregnated with a lotion.

Another object of the present invention is to provide a lotion applicator that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the lotion applicator.

FIG. 2 shows a perspective view of an alternate embodiment of the lotion applicator.

FIG. 3 shows a perspective view of another alternate embodiment of the lotion applicator.

FIG. 4 shows a perspective view of the lotion applicator in use.

### DETAILED DESCRIPTION OF THE INVENTION

Reference is made herein to the attached drawings. Like reference numerals are used throughout the drawings to depict like or similar elements of the lotion applicator. For the purposes of presenting a brief and clear description of the present invention, the preferred embodiment will be discussed as used for allowing to user to more easily apply lotion to a user's body. The figures are intended for representative purposes only and should not be considered to be limiting in any respect.

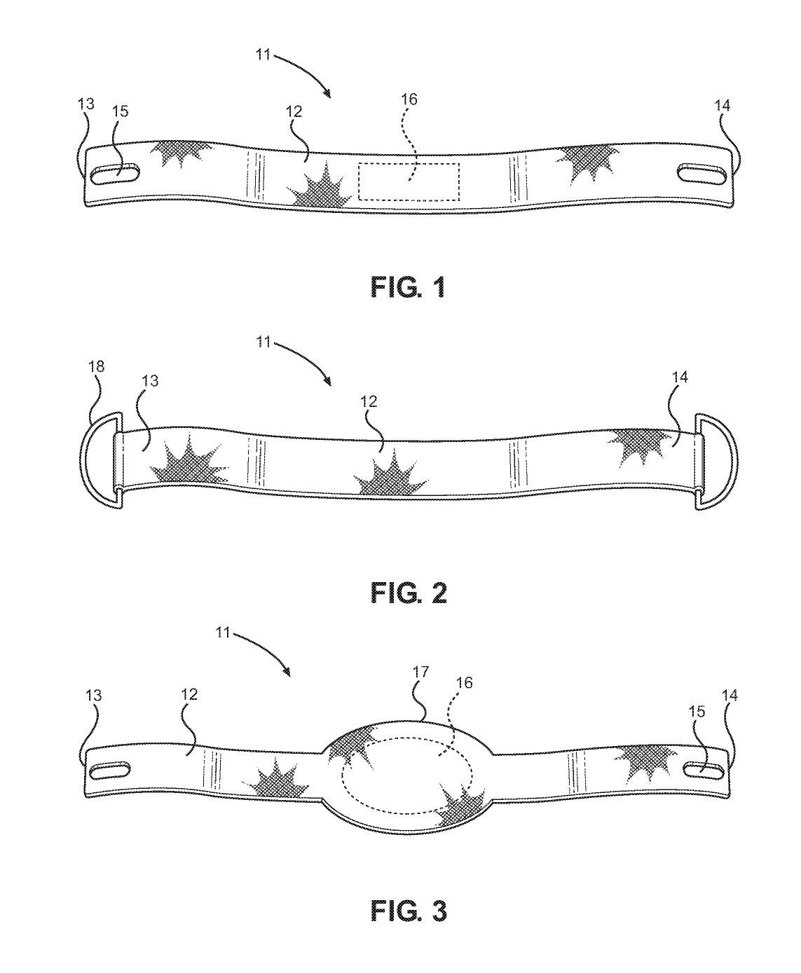

Referring now to FIGS. 1 and 2, there are shown views of alternate embodiments of the lotion applicator. The lotion applicator 11 comprises an elongated strap 12 having a first end 13 and a second end 14. The elongated strap 12 is shown as having a rectangular configuration, however, in alternate embodiments, the elongated strap may have alternative shapes or configurations. In some embodiments, the elongated strap 12 comprises a rope, or a bundle of wound or braided fibers capable of supporting a lotion thereon.

The elongated strap 12 is composed of a fabric material so that it can hold and support a lotion thereon. Further, the elongated strap 12 is flexible so that it can contour to the shape of the user's body and rest flush against the user's body in order to apply lotion evenly. Preferably, the elongated strap 12 is composed of a non-woven cotton material that is smooth to provide comfort and that is durable so as to prevent the elongated strap 12 from tearing or becoming damaged. The elongated strap 12 is composed of a low absorbency material so as to reduce saturation of the elongated strap 12 with the lotion, as saturation of the elongated strap 12 with lotion would result in waste. In alternate embodiments, the elongated strap 12 is composed of other materials, such as a flexible plastic material, among other materials suitable for supporting lotion thereon for application to a user's body.

A central portion 16 of the elongated strap 12 is adapted to receive lotion thereon. In some embodiments, the central portion 16 comprises a pad on which the user may deposit the lotion and that helps to indicate to the user where lotion should be positioned. In alternate embodiments, the central portion 16 comprises a lotion impregnated therein so that the user does not have to manually deposit lotion onto the elongated strap 12.

The first end 13 of the elongated strap 14 comprises a handle 15, and the second end of the elongated strap 12 also comprise a handle 15. In operation, the user can hold one handle 15 in each hand so as to maintain the elongated strap 12 in a stretched configuration. In FIG. 1, the handles 15 are shown as apertures disposed on the first end 13 and the second end 14 of the elongated strap 12. The user can insert his or her fingers through the apertures in order to grasp the ends of the elongated strap 12. In the alternate embodiment of FIG. 2, the handles comprise rings 18 that are partially affixed to the first end 13 and second end 14 of the elongated strap 12. The rings 18 may provide a more stable support for a user to grasp. In the illustrated embodiment, the rings 18 are shown as having a D-shaped configuration with a central opening. In alternate embodiments, the rings 18 may have a circular shape, among others.

Referring now to FIG. 3, there is shown a perspective view of another alternate embodiment of the lotion applicator. In some embodiments, the elongated strap 12 comprises a widened central region 17. The widened central region 17 provides more surface area so that the lotion applicator 11 can contact a larger portion of a user's body to apply lotion more efficiently. In the illustrated embodiment, the widened central region 17 comprises an oval or elliptical shape. In operation, the user can deposit lotion onto the widened central region 17, and can then position the widened central region against a body part so as to apply the lotion thereto. In some embodiments, the widened central region 17 comprises a pad 16 thereon on which the lotion can be positioned.

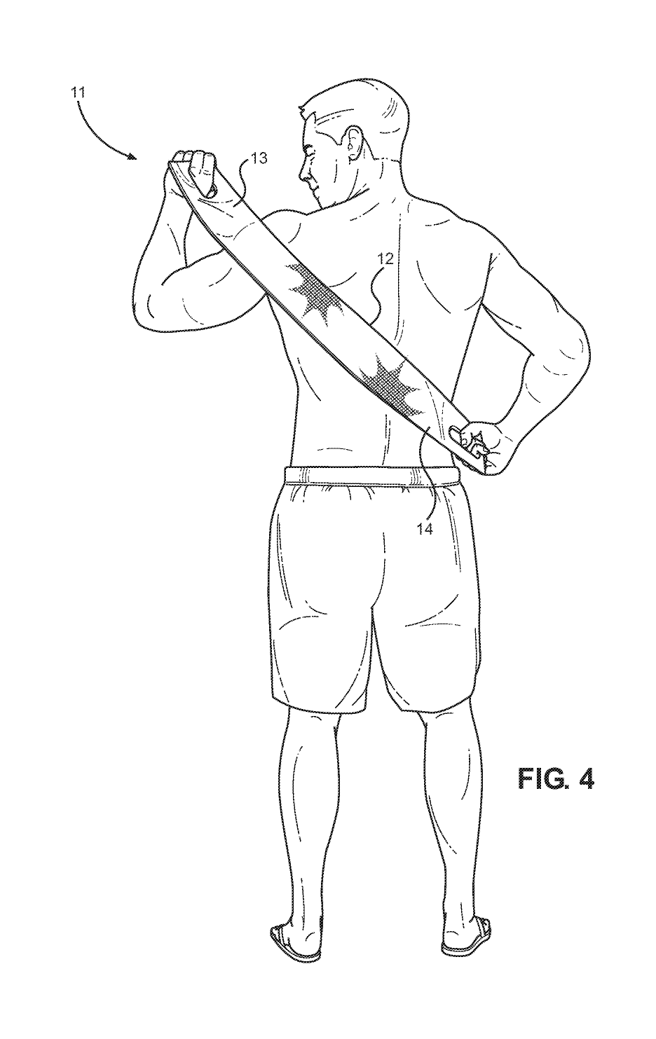

Referring now to FIG. 4, there is shown a perspective view of the lotion applicator in use. In operation, the user can apply a lotion to the elongated strap 12 that the user desires to apply to his or her body. The user can grasp a handle on each end 13, 14 of the elongated strap 12 in each hand. The user can place the elongated strap in contact with a body part, such as the user's back, for applying the lotion thereto. The elongated strap 12 fully contacts the user's body so as to evenly apply the lotion thereon. The user can move the elongated strap 12 in a back and forth motion in order to apply and rub the lotion on the elongated strap 12 into the user's skin. In this way, the lotion applicator allows the user to easily apply lotion directly to hard to reach areas, such as the user's back, the back of the user's legs, among various other areas.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A lotion applicator, comprising:
an elongated strap having a first end and a second end;
a handle disposed on each of the first end and the second end;
wherein the elongated strap is adapted to receive a lotion thereon for application to a body part of a user.

2) The lotion applicator of claim 1, wherein the elongated strap is composed of a non-woven cotton material.

3) The lotion applicator of claim 1, wherein the elongated strap comprises a rectangular configuration.

4) The lotion applicator of claim 1, wherein the elongated strap comprises a widened central region.

5) The lotion applicator of claim 1, wherein the handles comprise apertures disposed on the first end and the second end of the elongated strap.

6) The lotion applicator of claim 1, wherein the handles comprise a ring affixed to the first end and a ring affixed to the second end of the elongated strap.

7) The lotion applicator of claim 1, wherein the elongated strap is impregnated with a lotion.

8) The lotion applicator of claim 1, wherein the elongated strap comprises an absorbent pad on which a lotion is adapted to be positioned.

9) A lotion applicator, comprising:
an elongated strap having a first end and a second end, wherein the elongated strap comprises a rectangular configuration, and wherein the elongated strap is adapted to receive a lotion thereon for applicator to a body of a user.
a first handle disposed on the first end of the elongated strap and a second handle disposed on the second end of the elongated strap, wherein the first handle is adapted to be held in a first hand of a user and the second handle is adapted to be held in the second hand of the user.

10) The lotion applicator of claim 9, wherein the elongated strap comprises a rope.
